(19) United States
(12) Patent Application Publication
(10) Pub. No.: US 2024/0180799 A1
(43) Pub. Date: Jun. 6, 2024

(54) Oral Care Compositions Containing Stannous Ion Source

(71) Applicant: Colgate-Palmolive Company, New York, NY (US)

(72) Inventors: Tatiana BRINZARI, Franklin Park, NJ (US); Chi-Yuan CHENG, Hillsborough, NJ (US); Zhigang HAO, Bridgewater, NJ (US); Long PAN, Somerset, NJ (US); Cristina CASTRO, Piscataway, NJ (US); Viktor DUBOVOY, Basking Ridge, NJ (US)

(73) Assignee: Colgate-Palmolive Company, New York, NY (US)

(21) Appl. No.: 18/529,111

(22) Filed: Dec. 5, 2023

Related U.S. Application Data
(60) Provisional application No. 63/430,230, filed on Dec. 5, 2022.
(60) Provisional application No. 63/430,229, filed on Dec. 5, 2022.

(51) Int. Cl. A61K 8/21; A61K 8/19; A61K 8/24; A61K 8/34; A61K 8/365; A61K 8/44; A61K 8/49; A61Q 11/00

(52) U.S. Cl. CPC A61K 8/21; A61K 8/19; A61K 8/24; A61K 8/347; A61K 8/365; A61K 8/44; A61K 8/498; A61Q 11/00; A61K 2800/52; A61K 2800/522

(57) ABSTRACT

Oral care compositions comprising (i) a stannous ion source and (ii) a chelator or an antioxidant, as well as to methods of using these compositions are disclosed herein.

### CROSS-REFERENCE TO RELATED PATENT APPLICATIONS

This application claims the benefit of priority from U.S. Provisional Application No. 63/430,229, filed Dec. 5, 2022, and from U.S. Appl. No. 63/430,230, titled Oral Care Compositions Containing Stannous Ion Source and filed Dec. 5, 2022, the contents for both of which are hereby incorporated herein in their entireties, for all purposes.

### BACKGROUND

Stannous fluoride (SnF₂) is well known for use in clinical dentistry with a history of therapeutic benefits dating back to the early 1950s. It has been reported to be an effective agent for treating various oral conditions and diseases including plaque, gingivitis, sensitivity, enamel decalcification, and periodontitis, among others. Stannous fluoride is widely used in commercial toothpastes and mouthwashes as an active ingredient delivering anticavity and antigingivitis benefits. However, it is well known that stannous fluoride is sensitive to oxidation. Stannous ion (Sn (II)) rapidly oxidizes to stannic ion (Sn (IV)) which is less bioactive. Therefore, maximizing the amount of tin in the stannous form (Sn (II)) is beneficial to providing these oral health benefits over the shelf life of the product.

Many efforts have been dedicated to suppress Sn(II) oxidation and achieve stable stannous formulations. One approach to suppressing Sn(II) oxidation is to use KNO₃ as a stabilizing agent (US 2013039867). It was found that at low pH, SnF₂ solution becomes more stable and less prone to oxidation when KNO₃ salt is added to SnF₂. Although several methods of stabilizing stannous ion have been known in the art, both maximization and stabilization of tin in the stannous form (tin II) in oral care formulations has remained an ongoing challenge.

Accordingly, there exists a need for stabilized stannous formulations with improved oral health benefits.

### BRIEF SUMMARY

The present disclosure provides oral care compositions, e.g., toothpaste or mouthwash, comprising (i) a stannous ion source (e.g., stannous fluoride) and (ii) a chelator or an antioxidant. In some embodiments, the chelator is selected from citrate and EDTA (ethylenediaminetetraacetic acid). In some embodiments, the antioxidant is selected from quercetin and catechol. In some embodiments, pH of the composition is from 6.5 to 7.5, e.g., from 6.6 to 7.4, from 6.7 to 7.3, from 6.8 to 7.2, from 6.9 to 7.1, or about 7.0. For example, the oral care composition may preferably be formulated to have a pH of about 5.5 to about 9, about 5.5 to about 8.5, about 5.5 to about 8, about 5.5 to about 7.5, about 5.5 to about 7, about 5.5 to about 6.5, about 5.5 to about 6; from about 6 to about 9, about 6 to about 8.5, about 6 to about 8, about 6 to about 7.5, about 6 to about 7, about 6 to about 6.5; from about 6.5 to about 9, about 6.5 to about 8.5, about 6.5 to about 8, about 6.5 to about 7.5, about 6.5 to about 7; from about 7 to about 9, about 7 to about 8.5, about 7 to about 8, about 7 to about 7.5; from about 7.5 to about 9, about 7.5 to about 8.5, about 7.5 to about 8; from about 8 to about 9, about 8 to about 8.5, or any range or subrange thereof.

The oral care composition may be formulated to have a molar ratio of the stannous ion source to the chelator (e.g. citrate and/or EDTA) of about 2:1 to about 1:3, about 2:1 to about 1:2.5, about 2:1 to about 1:2, about 2:1 to about 1:1.5, about 2:1 to about 1:1; about 1.5:1 to about 1:3, about 1.5:1 to about 1:2.5, about 1.5:1 to about 1:2, about 2:1 to about 1:1.5, about 1.5:1 to about 1:1; about 1:1 to about 1:3, about 1:1 to about 1:2.5, about 1:1 to about 1:2, about 1:1 to about 1:1.5, or any range or subrange thereof.

In some embodiments, the oral care composition comprises a stannous ion source and a chelator selected from citrate and EDTA. In some embodiments, the composition further comprises potassium nitrate. In some embodiments, the chelator is citrate and the molar ratio of stannous ion source:citrate:potassium nitrate is 1:1.5-2.5:0.5-1.5, e.g., 1:1.7-2.3:0.7-1.3, 1:1.8-2.2:0.8-1.2, 1:1.9-2.1:0.9-1.1, or about 1:2:1. In other embodiments, the chelator is EDTA and the molar ratio of stannous ion source:EDTA:potassium nitrate is 1:0.5-1.5:0.5-1.5, e.g., 1:0.7-1.3:0.7-1.3, 1:0.8-1.2:0.8-1.2, 1:0.9-1.1:0.9-1.1, or about 1:1:1. In some embodiments, the chelator is citrate and the molar ratio of stannous ion:citrate ion:nitrate ion is 1:1.5-2.5:0.5-1.5, e.g., 1:1.7-2.3:0.7-1.3, 1:1.8-2.2:0.8-1.2, 1:1.9-2.1:0.9-1.1, or about 1:2:1. In other embodiments, the chelator is EDTA and the molar ratio of stannous ion:EDTA ion:nitrate ion is 1:0.5-1.5:0.5-1.5, e.g., 1:0.7-1.3:0.7-1.3, 1:0.8-1.2:0.8-1.2, 1:0.9-1.1:0.9-1.1, or about 1:1:1.

In some embodiments, the oral care composition comprises a stannous ion source and an antioxidant selected from quercetin and catechol. In some embodiments, the composition further comprises tetrasodium pyrophosphate (TSPP). In some embodiments, the molar ratio of stannous TSPP:antioxidant is 1:0.5-1.5:0.2-0.4, e.g., 1:0.7-1.3:0.25-0.35, 1:0.8-1.2:0.28-0.32, 1:0.9-1.1:0.29-0.31, or about 1:1:0.3.

The present disclosure further provide a methods comprising applying an effective amount of an oral care composition as disclosed herein to the oral cavity, e.g., by brushing, to a subject in need thereof, to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity.

The present disclosure further encompasses the use of a chelator or an antioxidant in an oral care composition comprising a stannous ion source for increasing the stability of stannous ion in the composition.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF THE DRAWINGS

FIG. 1 shows the infrared absorption spectra (1000-1800 cm¹ range) of Sn(II)-EDTA complex in water along with EDTA and KNO₃ reference solutions at pH=7. Spectra are offset for clarity.

FIG. 2 displays the vibrational spectra of fresh Sn(II)-EDTA complex with or without KNO₃ salt and Sn(II)-EDTA complex with or without KNO₃ salt after 2 weeks of accelerated aging a temperature of 60° C. Spectra are offset for clarity.

FIG. 3A shows the relative amount of Sn(II) in the samples (SnF₂+citrate with or without KNO₃) upon aging. FIG. 3B shows the relative amount of Sn(II) in the samples (SnF₂+EDTA with or without KNO₃) upon accelerated aging at a temperature of 60° C.

FIG. 4 depicts a graph showing head-space O₂ consumption monitored indirectly through differential pressure changes in solutions containing stannous fluoride and sodium citrate with KNO₃ or without KNO₃.

FIGS. 5A and 5B show the crystal structure of Sn(II)-citrate. FIG. 5A depicts the Sn(II) cluster and FIG. 5B depicts the coordination of the citrate molecule.

FIG. 6 shows PXRD of Sn-Citrate samples soaked in deionized water upon aging at a temperature of 60° C.

FIG. 7 displays PXRD of Sn-Citrate samples soaked in KNO₃ solution upon aging at a temperature of 60° C.

FIG. 8 shows the FTIR spectra (850-1225 cm⁻¹) of solutions containing SnF₂ and TSPP with and without antioxidant at fresh and after two weeks of accelerated gaining at a temperature of 60° C. Water spectrum was subtracted from each individual curve. The spectra are offset for clarity. The vertical dashed lines guide the eye.

FIG. 9 shows the amount of Sn(II) present in the samples (SnF₂-TSPP, SnF₂-TSPP-Quercetin, SnF₂-TSPP-Catechol) after 2 week of accelerated aging at a temperature of 60° C. with or without the antioxidants.

FIG. 10 represents ¹¹⁹Sn NMR spectra of SnF₂-TSPP-quercetin and SnF₂-TSPP-catechol upon aging at 60° C.

FIG. 11 shows Sn(II) % of SnF₂-TSPP-quercetin and SnF₂-TSPP-catechol upon aging at 60° C.

FIG. 12 is a graph showing the pressure differential of a non-limiting example composition and a comparative composition undergoing stannous oxidation reactions according to aspects of the disclosure.

FIG. 13 is a graph showing the pressure differential of a non-limiting example composition and a comparative composition undergoing stannous oxidation reactions according to aspects of the disclosure.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The present invention provides, in an aspect, an oral care composition (Composition 1.0), for example toothpaste or mouthwash, that comprises (i) a stannous ion source and (ii) a chelator or an antioxidant.

For example, the invention includes:
1.1. Composition 1.0, wherein the oral care composition comprises a chelator selected from a citrate and EDTA (ethylenediaminetetraacetic acid). The chelator may be present in the oral care composition in an amount from about 0.1 to about 4 wt. %, based on the total weight of the oral care composition. For instance, the total amount of about 0.1 to about 4 wt. %, about 0.1 to about 3 wt. %, about 0.1 to about 2.5 wt. %, about 0.1 to about 2 wt. %, about 0.1 to about 1.5 wt. %, about 0.1 to about 1 wt. %, about 0.1 to about 0.5 wt. %; from about 0.5 to about 4 wt. %, about 0.5 to about 3 wt. %, about 0.5 to about 2.5 wt. %, about 0.5 to about 2 wt. %, about 0.5 to about 1.5 wt. %, about 0.5 to about 1 wt. %; from about 1 to about 4 wt. %, about 1 to about 3 wt. %, about 1 to about 2.5 wt. %, about 1 to about 2 wt. %, about 1 to about 1.5 wt. %; from about 1.5 to about 4 wt. %, about 1.5 to about 3 wt. %, about 1.5 to about 2.5 wt. %, about 1.5 to about 2 wt. %; from about 2 to about 4 wt. %, about 2 to about 3 wt. %, about 2 to about 2.5 wt. %; from about 2.5 to about 4 wt. %, about 2.5 to about 3 wt. %, about 3 to about 4 wt. %, based on the total weight of the oral care composition.
1.2. Composition 1.0 or 1.1, wherein the oral care composition comprises potassium nitrate. The potassium nitrate may be present in an amount from about 0.1 to about 5 wt. %, based on the total weight of the oral care composition.
1.3. Any of the preceding oral care compositions, wherein the chelator is a citrate. The citrate may be selected from trisodium citrate, disodium citrate, mono citrate, stannous citrate, potassium citrate, and a combination thereof. Additionally or alternatively, the citrate may comprise citric acid. In at least one embodiment, the citrate is selected from any salt of citric acid.
1.4. Composition 1.3, wherein the oral care composition has a molar ratio of stannous citrate:potassium nitrate is 1:1.5-2.5:0.5-1.5, optionally wherein the molar ratio of stannous citrate:potassium nitrate is 1:1.7-2.3:0.7-1.3, e.g., 1:1.8-2.2:0.8-1.2, 1:1.9-2.1:0.9-1.1, or about 1:2:1.
1.5. Any of the Compositions 1.1 to 1.4, wherein the oral care composition further comprises an antioxidant, optionally wherein the antioxidant is selected from quercetin and catechol. Antioxidant may comprise one or more Vitamin C, such as those described herein. In some embodiments, the antioxidants consist of one or more Vitamin C, such as those disclosed herein. The antioxidant(s) may be present in the oral care composition in an amount from about 0.05 to about 5 wt. %, based on the total weight of the oral care composition. In some embodiments, the total amount of antioxidant(s) is from about 0.05 to about 0.1 wt. %, based on the total weight of the oral care composition.
1.6. Composition 1.5, wherein the oral care composition has a molar ratio of stannous citrate:potassium nitrate:antioxidant is 1:1.5-2.5:0.5-1.5:0.2-0.4, optionally wherein the molar ratio of stannous:citrate:potassium nitrate:antioxidant is 1:1.7-2.3:0.7-1.3:0.25-0.35, e.g., 1:1.8-2.2:0.8-1.2:0.28-0.32, 1:1.9-2.1:0.9-1.1:0.29-0.31, or about 1:2:1:0.3
1.7. Compositions 1.1 or 1.2, wherein the chelator is EDTA.
1.8. Composition 1.7, wherein the oral care composition has a molar ratio of stannous EDTA:potassium nitrate is 1:0.5-1.5:0.5-1.5, optionally wherein the molar ratio of stannous:citrate:potassium nitrate is 1:0.7-1.3:0.7-1.3, e.g., 1:0.8-1.2:0.8-1.2, 1:0.9-1.1:0.9-1.1, or about 1:1:1.
1.9. Compositions 1.7 or 1.8, wherein the oral care composition further comprises an antioxidant, optionally wherein the antioxidant is selected from quercetin and catechol.
1.10. Composition 1.9, wherein the oral care composition has a molar ratio of stannous EDTA:potassium nitrate:antioxidant is 1:0.5-1.5:0.5-1.5:0.2-0.4, optionally wherein the molar ratio of stannous:citrate:potassium nitrate:antioxidant is 1:0.7-1.3:0.7-1.3:0.25-0.35, e.g., 1:0.8-1.2:0.8-1.2:0.28-0.32, 0.9-1.1:0.9-1.1:0.9-1.1:0.29-0.31, or about 1:2:1:0.3.
1.11. Any of Compositions 1.1-1.10, wherein the oral care composition does not contain any polyphosphate or pyrophosphate.
1.12. Composition 1.0, wherein the oral care composition comprises an antioxidant selected from quercetin and catechol.
1.13. Composition 1.12, wherein the oral care composition comprises tetrasodium pyrophosphate (TSPP). The tetrasodium pyrophosphate may be present in the oral care composition in an amount from about 0.1 to about 5 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.
1.14. Composition 1.12 or 1.13, wherein the molar ratio of stannous:TSPP:antioxidant is 1:0.5-1.5:0.2-0.4, optionally wherein the molar ratio of stannous:TSPP:antioxidant is 1:0.7-1.3:0.25-0.35, e.g., 1:0.8-1.2:0.28-0.32, 1:0.9-1.1:0.29-0.31, or about 1:1:0.3.
1.15. Any of Compositions 1.12 to 1.14, wherein the oral care composition comprises potassium nitrate.
1.16. Any of Compositions 1.12 to 1.14, wherein the oral care composition does not contain potassium nitrate.
1.17. Any of the preceding compositions, wherein the stannous ion source is selected from the group consisting of stannous fluoride, stannous gluconate, stannous phosphate, stannous pyrophosphate, stannous acetate, stannous sulfate, stannous chloride, and a combination thereof.
1.18. Any of the preceding compositions, wherein the stannous ion source is stannous fluoride.
1.19. Composition 1.18, wherein the composition further comprises a stannous ion source which is not stannous fluoride.
1.20. Any of the preceding compositions, wherein the stannous ion source is present in an amount of from 0.01% to 10%, e.g., from 0.1% to 5%, from 1% to 5%, from 1.5 to 4%, from 0.1% to 1%, from 0.1% to 0.2%, from 0.2% to 0.8%, from 0.2% to 0.5%, from 0.3% to 0.6%, or from 0.4% to 0.5%, by weight, based on the total weight of the oral care composition.
1.21. Any of the preceding compositions, wherein the oral care composition further comprises a zinc source. The zinc source may be a zinc ion source.
1.22. Composition 1.21, wherein the zinc source (e.g., a zinc ion source) is selected from the group consisting of zinc oxide, zinc sulfate, zinc chloride, zinc citrate, zinc lactate, zinc gluconate, zinc malate, zinc tartrate, zinc carbonate, zinc phosphate, and a combination thereof.
1.23. Combination 1.21 or 1.22, wherein the zinc source is present an amount of from 0.01% to 5%, e.g., 0.1% to 4%, or 0.5% to 3%, by weight, based on the total weight of the oral care composition. For example, the amount of zinc source present in the oral care composition may be from about 0.1 to about 5 wt. %, about 0.1 to about 4 wt. %, about 0.1 to about 3 wt. %, about 0.1 to about 2 wt. %, about 0.1 to about 1 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %, about 3 to about 5 wt. %, or any range or subrange thereof, based on the total weight of the oral care composition.
1.24. Any of Compositions 1.21 to 1.23, wherein the oral care composition comprises zinc oxide.
1.25. Composition 1.24, wherein zinc oxide is present in an amount of from 0.5% to 2%, e.g., from 0.5% to 1.5%, from 0.8% to 1.3%, from 1% to 1.2%, from 1.1% to 1.3%, about 1%, or about 1.2% by weight, based on the total weight of the oral care composition.
1.26. Any of Compositions 1.21 to 1.23, wherein the oral care composition comprises zinc oxide and zinc citrate.
1.27. Composition 1.26, wherein zinc oxide is present in an amount of from 0.5% to 2%, e.g., from 0.5% to 1.5%, from 0.8% to 1.3%, from 1% to 1.2%, from 1.1% to 1.3%, about 1%, or about 1.2% by weight of the composition and zinc citrate is present in an amount of from 0.1% to 1%, from 0.25% to 0.75%, from 0.3% to 0.6%, about 0.5%, by weight, based on the total weight of the oral care composition.
1.28. Composition 1.27, wherein zinc oxide is present in an amount of about 1%, by weight, based on the total weight of the oral care composition and zinc citrate is present in an amount of about 0.5%, by weight, based on the total weight of the oral care composition.
1.29. Any of the preceding compositions, wherein the oral care composition comprises zinc phosphate.
1.30. Composition 1.29, wherein zinc phosphate is present in an amount of from 0.5% to 2%, e.g., from 0.5% to 1.5%, from 0.8% to 1.3%, from 1% to 1.2%, from 1.1% to 1.3%, about 1%, or about 1.2%, by weight, based on the total weight of the oral care composition.
1.31. Any of the preceding compositions, wherein the oral care composition comprises a fluoride ion source is selected from stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and a combination thereof.
1.32. Composition 1.31, wherein the fluoride ion source is stannous fluoride.
1.33. Composition 1.32, wherein the oral care composition further comprises a fluoride ion source which is not stannous fluoride.
1.34. The composition of composition 1.31 to 1.33, wherein the oral care composition comprises fluoride ion sources in amounts sufficient to supply 25 ppm to 5,000 ppm of fluoride ions, generally at least 500 ppm, e.g., 500 to 2000 ppm, e.g., 1000 ppm to 1600 ppm, e.g., 1450 ppm.
1.35. Any of the preceding compositions, wherein the oral care composition comprises a basic amino acid in free or salt form.
1.36. Composition 1.35, wherein the basic amino acid comprises one or more of arginine, lysine, citrulline, ornithine, creatine, histidine, diaminobutyric acid, diaminopropionic acid, salts thereof, or combinations thereof.
1.37. Composition 1.35 or 1.36, wherein the basic amino acid has the L-configuration.
1.38. Any of Compositions 1.35 to 1.37, wherein the basic amino acid is present in an amount of from 1% to 15%, e.g., from 1% to 10%, from 1% to 5%, from 1% to 3%, from 1% to 2%, from 1.2% to 1.8%, from 1.4% to 1.6%, or about 1.5% by weight, based on the total weight of the oral care composition, being calculated as free base form.
1.39. Any of Compositions 1.35-1.38, wherein the basic amino acid comprises arginine.
1.40. Composition 1.39, wherein the basic amino acid comprises arginine bicarbonate, arginine phosphate, arginine sulfate, arginine hydrochloride or combinations thereof, optionally wherein the basic amino acid is arginine bicarbonate.
1.41. Any of the preceding compositions, wherein the oral care composition comprises one or more thickeners, for example thickening silicas.
1.42. Any of the preceding compositions, wherein the oral care composition comprises a foaming agent, for example a betaine, for example cocamidopropyl betaine.
1.43. Any of the preceding compositions, wherein the oral care composition comprises ingredients selected from one or more of buffering agents, humectants, surfactants, gum strips or fragments, breath fresheners, flavoring, fragrance, coloring, antibacterial agents, whitening agents, agents that interfere with or prevents bacterial attachment, calcium sources, and potassium salts.
1.44. Any of the preceding compositions, wherein the pH of the oral care composition is from 6.5 to 7.5, optionally wherein pH of the composition is from 6.6 to 7.4, e.g., from 6.7 to 7.3, from 6.8 to 7.2, from 6.9 to 7.1, or about 7 For example, the oral care composition may preferably be formulated to have a pH of about 5.5 to about 9, about 5.5 to about 8.5, about 5.5 to about 8, about 5.5 to about 7.5, about 5.5 to about 7, about 5.5 to about 6.5, about 5.5 to about 6; from about 6 to about 9, about 6 to about 8.5, about 6 to about 8, about 6 to about 7.5, about 6 to about 7, about 6 to about 6.5; from about 6.5 to about 9, about 6.5 to about 8.5, about 6.5 to about 8, about 6.5 to about 7.5, about 6.5 to about 7; from about 7 to about 9, about 7 to about 8.5, about 7 to about 8, about 7 to about 7.5; from about 7.5 to about 9, about 7.5 to about 8.5, about 7.5 to about 8; from about 8 to about 9, about 8 to about 8.5, or any range or subrange thereof.
1.45. Any of the preceding compositions, wherein the composition is a dentifrice, a toothpaste, a gel, a mouthwash, a mouth rinse, a powder, a cream, a strip, a gum, bead, film, or floss.
1.46. Composition 1.45, wherein the composition is a toothpaste.
1.47. Composition 1.45, wherein the composition is a gel.
1.48. Composition 1.45, wherein the composition is a mouthwash.
1.49. Any of the preceding compositions for use to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity.

The composition of the present invention comprises a stannous ion source. Stannous ion sources are well known in the art and may be incorporated into the compositions of the present invention. In some embodiments, the stannous ion source is selected from the group consisting of stannous fluoride, stannous gluconate, stannous phosphate, stannous pyrophosphate, stannous acetate, stannous sulfate, stannous chloride and a combination thereof. In some embodiments, the stannous ion source is present in an amount of from 0.01% to 10%, e.g., from 0.1% to 5%, from 1% to 5%, from 1.5 to 4%, from 0.1% to 1%, from 0.1% to 0.2%, from 0.2% to 0.8%, from 0.2% to 0.5%, from 0.3% to 0.6%, or from 0.4% to 0.5%, by weight, based on the total weight of the oral care composition. In certain embodiments, the stannous ion source is stannous fluoride. In some embodiments, stannous fluoride is present in an amount of from 0.01% to 10%, e.g., from 0.5% to 7%, from 1% to 5%, from 1.5 to 4%, from 0.1% to 1%, from 0.1% to 0.2 from 0.2% to 0.8%, from 0.2% to 0.5%, from 0.3% to 0.6%, or from 0.4% to 0.5%, by weight, based on the total weight of the oral care composition. In some embodiments, the composition may contain other stannous ion source which is not stannous fluoride.

Stannous ion (Sn (II)) rapidly oxidizes to stannic ion (Sn (IV)) which is less bioactive. In the present invention, it has been found that a high proportion of tin is present in the stannous form (Sn (II)) with aging when citrate or EDTA is added to a composition having a neural pH which contains a stannous ion source and potassium nitrate. Without intending to be bound to any theory, it is believed that stannous ions are chelated by citrate or EDTA and potassium nitrate stabilizes the Sn(II)-chelator complex at and/or near neutral pH. It has also been found that a high proportion of tin is present in the stannous form (Sn (II)) with aging when an antioxidant, such as quercetin and catechol, is added to an oral care composition having a neural pH or near neutral pH which contains a stannous ion source and tetrasodium pyrophosphate (TSPP). Without intending to be bound to any theory, it is believed that stannous ions are chelated by TSPP and antioxidants suppress the oxidation of stannous ion and stabilize the Sn(II)-pyrophosphate complex at or near neutral pH.

In some embodiments, the oral care composition comprises a stannous ion source, potassium nitrate, and a chelator. The chelator may be selected from a citrate and EDTA. The chelator is present in an effective amount to stabilize stannous ion. When citrate is used as a chelator, the molar ratio of stannous:citrate:potassium nitrate may be 1:1.5-2.5:0.5-1.5, e.g., 1:1.7-2.3:0.7-1.3, 1:1.8-2.2:0.8-1.2, 1:1.9-2.1:0.9-1.1, or about 1:2:1. When EDTA is used as a chelator, the molar ratio of stannous:EDTA:potassium nitrate may be 1:0.5-1.5:0.5-1.5, e.g., 1:0.7-1.3:0.7-1.3, 1:0.8-1.2:0.8-1.2, 1:0.9-1.1:0.9-1.1, or about 1:1:1. In some embodiments, the oral care composition comprising a stannous ion source, potassium nitrate, and a chelator selected from citrate and EDTA does not contain any polyphosphate or pyrophosphate.

In some embodiments, the oral care composition comprises a stannous ion source, tetrasodium pyrophosphate, and an antioxidant agent. Antioxidants are compounds that have the ability to scavenge free radicals and thus slow down or suppress the oxidation processes by disrupting the radical chain reactions and inhibiting the formation of strong oxidation species. In some embodiments, the antioxidant is selected from quercetin and catechol. The one or more antioxidant(s) may comprise a Vitamin C, such as any of the Vitamin Cs disclosed above. The antioxidant may comprise one or more Vitamin C, such as those described above. In some embodiments, the antioxidants consist of one or more Vitamin C, such as those disclosed herein. For example, the antioxidants may consists of one or more of butylated hydroxytoluene (BHT), butylated hydroxyanisole (BHA), ascorbic acid, calcium ascorbate, calcium 1-ascorbate dihydrate, magnesium ascorbate, potassium ascorbate, magnesium L-ascorbyl phosphate, L-ascorbic acid 2-phosphate sesquimagnesium salt hydrate, (+) sodium L-ascorbate, dehydro-1-(+)-ascorbic acid dimer, sodium ascorbyl phosphate, ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbyl glucoside, ascorbic acid polypeptide, ethyl ascorbyl ether, ascorbyl ethyl silanol pectinate, and a combination of two or more thereof. The Vitamin C may be an ascorbate. The ascorbate may be selected from calcium ascorbate, calcium 1-ascorbate dihydrate, magnesium ascorbate, potassium ascorbate, magnesium L-ascorbyl phosphate (also referred to as: magnesium ascorbate phosphate or ascorbic acid phosphate magnesium salt), and a combination of two or more thereof.

In some embodiments, the antioxidant is selected from ascorbyl phosphate, ascorbate (or ascorbic acid), butylated hydroxytoluene (BHT), and butylated hydroxyanisole (BHA). In some embodiments, the antioxidant is selected from an ascorbyl phosphate, an ascorbate, and acids thereof (such as, ascorbic acid). The antioxidants may be in free or salt form. In some embodiment, ascorbyl phosphate is sodium ascorbyl phosphate and ascorbate is sodium ascorbate. In certain embodiments, the antioxidant is ascorbyl phosphate, e.g., sodium ascorbyl phosphate.

The antioxidant(s) may be present in the oral care composition in an amount from about 0.05 to about 5 wt. %, based on the total weight of the oral care composition. For example, the total amount of antioxidant(s) in the oral care composition may be from about 0.05 to about 5 wt. %, about 0.05 to about 4 wt. %, about 0.05 to about 3 wt. %, about 0.05 to about 2 wt. %, about 0.05 to about 1.5 wt. %, about 0.05 to about 1.2 wt. %, about 0.05 to about 1 wt. %, about 0.05 to about 0.8 wt. %, about 0.05 to about 0.6 wt. %; from about 0.1 to about 5 wt. %, about 0.1 to about 4 wt. %, about 0.1 to about 3 wt. %, about 0.1 to about 2 wt. %, about 0.1 to about 1.5 wt. %, about 0.1 to about 1.2 wt. %, about 0.1 to about 1 wt. %, about 0.1 to about 0.8 wt. %, about 0.1 to about 0.6 wt. %; from about 0.5 to about 5 wt. %, about 0.5 to about 4 wt. %, about 0.5 to about 3 wt. %, about 0.5 to about 2 wt. %, about 0.5 to about 1.5 wt. %, about 0.5 to about 1.2 wt. %, about 0.5 to about 1 wt. %, about 0.5 to about 0.8 wt. %; from about 0.8 to about 5 wt. %, about 0.8 to about 4 wt. %, about 0.8 to about 3 wt. %, about 0.8 to about 2 wt. %, about 0.8 to about 1.5 wt. %, about 0.8 to about 1.2 wt. %, about 0.8 to about 1 wt. %; from about 1 to about 5 wt. %, about 1 to about 4 wt. %, about 1 to about 3 wt. %, about 1 to about 2 wt. %, about 1 to about 1.5 wt. %, about 1 to about 1.2 wt. %; from about 1.2 to about 5 wt. %, about 1.2 to about 4 wt. %, about 1.2 to about 3 wt. %, about 1.2 to about 2 wt. %, about 1.2 to about 1.5 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the oral care composition. In some embodiments, the total amount of antioxidant(s) is from about 0.05 to about 0.1 wt. %, based on the total weight of the oral care composition.

The chelator is present in an effective amount to solubilize stannous ion. In some embodiments, the molar ratio of stannous:TSPP:antioxidant is 1:0.5-1.5:0.2-0.4, e.g., 1:0.7-1.3:0.25-0.35, 1:0.8-1.2:0.28-0.32, 1:0.9-1.1:0.29-0.31, or about 1:1:0.3. In some embodiments, the oral care composition comprises a stannous ion source, tetrasodium pyrophosphate, an antioxidant agent, and potassium nitrate. In other embodiments, the oral care composition comprises a stannous ion source, tetrasodium pyrophosphate, wherein the oral care composition does not contain potassium nitrate.

The oral care compositions typically comprises one or more nitrate ion source (e.g., potassium nitrate). The nitrate ion source(s) (e.g., potassium nitrate) may be present in the oral care composition in an amount from about 0.1 to about 5 wt. %, based on the total weight of the oral care composition. In some instances, the amount of nitrate ion present in the oral care composition may be from about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The oral care compositions may be formulated to have a molar ratio of nitrate ions to stannous ions, both measured as free ions, that is about 2:1 or less. For example, the oral care composition may have a molar ratio of nitrate ions to stannous ions, both measured as free ions, of from about 0.5:1 to about 2:1, about 0.5:1 to about 1.8:1, about 0.5:1 to about 1.6:1, about 0.5:1 to about 1.4:1, about 0.5:1 to about 1.2:1, about 0.5:1 to about 1:1; from about 0.7:1 to about 2:1, about 0.7:1 to about 1.8:1, about 0.7:1 to about 1.6:1, about 0.7:1 to about 1.4:1, about 0.7:1 to about 1.2:1, about 0.7:1 to about 1:1; from about 0.9:1 to about 2:1, about 0.9:1 to about 1.8:1, about 0.9:1 to about 1.6:1, about 0.9:1 to about 1.4:1, about 0.9:1 to about 1.2:1, about 0.9:1 to about 1:1, or any range or subrange thereof. In some embodiments, the oral care composition is formulated to have a molar ratio of nitrate ions to stannous ions, both measured as free ions, of about 1:1.

The oral care composition of the present invention can be in the form of any oral care formulations, including dentifrice, toothpaste, gel, mouthwash, mouth rinse, powder, cream, strip, gum, bead, film, floss or any other known in the art. In some embodiments, the oral care composition is a toothpaste or gel. In other embodiments, the oral care composition is a mouthwash or mouth rinse.

The oral care composition of the present invention may be a single phase oral care composition. For example, all the components of the oral care composition may be maintained together with one another in a single phase and/or vessel. For example, all the components of the oral care composition may be maintained in a single phase, such as a single homogenous phase. In another embodiment, the oral care composition may be a multi-phase oral care composition.

The oral care composition of the present invention may contain an orally acceptable carrier. As used herein, an “orally acceptable carrier” refers to a material or combination of materials that are safe for use in the compositions of the invention, commensurate with a reasonable benefit/risk ratio. Such materials include but are not limited to, for example, water, humectants, ionic active ingredients, buffering agents, anticalculus agents, abrasive polishing materials, peroxide sources, alkali metal bicarbonate salts, surfactants, titanium dioxide, coloring agents, flavor systems, sweetening agents, antimicrobial agents, herbal agents, desensitizing agents, stain reducing agents, and mixtures thereof. Such materials are well known in the art and are readily chosen by one skilled in the art based on the physical and aesthetic properties desired for the compositions being prepared. In some embodiment, the orally acceptable carrier may include an orally acceptable solvent. Illustrative solvents may include, but are not limited to, one or more of ethanol, phenoxyethanol, isopropanol, water, cyclohexane, methyl glycol acetate, benzyl alcohol, or the like, or any mixture or combination thereof. In a particular embodiment, the orally acceptable solvent includes benzyl alcohol.

Water may be present in the oral care composition of the present invention. Water employed in the preparation of commercial oral care compositions should be deionized and free of organic impurities. Water may makes up the balance of the oral care compositions. In some instances, the total amount of water present in the oral care composition may be about 10% to about 90%, about 10% to about 80%, about 20% to about 60%, about 20% to 40%, about 10% to about 30%, about 20% to 30%, about 25% to 35%, about 70% to 90%, or about 80% to 90%, by weight, based on the total weight of the oral compositions. The total amount of water includes the free water which is added plus that amount which is introduced with other materials such as with sorbitol or any components of the oral care composition.

The oral care composition of the present invention may comprise a pH adjuster. For example, the oral care compositions may comprise an acid or base in an amount sufficient to adjust the pH of the oral care compositions. In some embodiments, pH of the oral care composition is neutral. The desired pH of the oral care composition may be from 6.5 to 7.5, e.g., from 6.6 to 7.4, from 6.7 to 7.3, from 6.8 to 7.2, from 6.9 to 7.1, or about 7.

In some embodiments, the oral care composition of the present invention comprises a fluoride ion source. Preferably, the fluoride ion source is stannous fluoride. In some embodiments, the oral care composition may contain other fluoride which is not stannous fluoride. Representative fluoride ion sources include, but are not limited to, sodium fluoride, potassium fluoride, sodium monofluorophosphate, sodium fluorosilicate, ammonium fluorosilicate, amine fluoride, ammonium fluoride, and combinations thereof. In some embodiments, the oral care composition may contain fluoride ion sources in amounts sufficient to supply 25 ppm to 5,000 ppm of fluoride ions, generally at least 500 ppm, e.g., 500 to 2000 ppm, e.g., 1000 ppm to 1600 ppm, e.g., 1450 ppm. Fluoride ion sources may be added to the oral care compositions of the invention at a level of 0.01% to 10%, e.g., 0.03% to 5%, or 0.1% to 1%, by weight, based on the total weight of the oral care composition.

The oral care composition may include one or more zinc source. The zinc source may be a zinc ion source. The zinc source can be a soluble or sparingly soluble compound of zinc with inorganic or organic counter ions. Examples of the zinc source include zinc oxide, zinc sulfate, zinc chloride, zinc citrate, zinc lactate, zinc gluconate, zinc malate, zinc tartrate, zinc carbonate, and zinc phosphate. In some embodiments, the zinc source is present in an amount of from 0.01% to 5%, e.g., 0.1% to 4%, or 1% to 3%, by weight, based on the total weight of the oral care composition.

In preferred embodiments, the composition comprises zinc oxide. Zinc oxide may be present in an amount of 0.5% to 2%, e.g., 0.5% to 1.5%, about 1% or about 1.2% by weight, based on the total weight of the oral care composition. In some embodiments, the oral care composition comprises zinc oxide and zinc citrate. The oral care compositions may comprise zinc oxide in an amount of 0.5% to 2%, e.g., 0.5% to 1.5%, about 1% or about 1.2%, by weight, based on the total weight of the oral care composition and zinc citrate in an amount of 0.1%-1%, 0.25-0.75%, about 0.5%, by weight, based on the total weight of the oral care composition. In some embodiments, the oral care compositions comprise zinc oxide in an amount of 1% by weight based on the total weight of the oral care composition and zinc citrate in an amount of 0.5% by weight based on the total weight of the oral care composition.

In some embodiments, the composition comprises zinc phosphate. In some embodiments, the composition may comprise zinc phosphate in an amount of 0.5% to 2%, e.g., 0.5% to 1.5%, about 1% or about 1.2%, by weight, based on the total weight of the oral care composition.

The oral care composition may comprise a basic amino acid in free or salt form. The basic amino acids which can be used in the compositions include not only naturally occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrulline, ornithine, creatine, histidine, diaminobutyric acid, diaminopropionic acid, salts thereof or combinations thereof. In a particular embodiment, the basic amino acids are selected from arginine, lysine, citrulline, and ornithine. The basic amino acids of the oral care composition may generally be present in the L-form or L-configuration. The basic amino acids may be provided as a salt of a di- or tri-peptide including the amino acid. In some embodiments, at least a portion of the basic amino acid present in the oral care composition is in the salt form. In some embodiments, the basic amino acid is arginine, for example, L-arginine, or a salt thereof. Arginine may be provided as free arginine or a salt thereof. For example, Arginine may be provided as arginine phosphate, arginine hydrochloride, arginine sulfate, arginine bicarbonate, or the like, and mixtures or combinations thereof. The basic amino acid may be provided as a solution or a solid. For example, the basic amino acid may be provided as an aqueous solution. In some embodiment, the amino acid includes or is provided by an arginine bicarbonate solution. For example, the amino acid may be provided by an about 40% solution of the basic amino acid, such as arginine bicarbonate or alternatively called as arginine carbamate. In some embodiments, the basic amino acid is present in an amount of from 1% to 15%, e.g., from 1% to 10%, from 1% to 5%, from 1% to 3%, from 1% to 2%, from 1.2% to 1.8%, from 1.4% to 1.6%, or about 1.5% by weight of the composition, being calculated as free base form.

The oral care composition may include other active ingredients. The active ingredients include, for example, anti-bacterial active agents, anti-tartar agents, anti-caries agents, anti-inflammatory agents, anti-sensitivity agents, enzymes, nutrients, and the like. Actives useful herein are optionally present in the oral care compositions in safe and effective amounts that are sufficient to have the desired therapeutic or prophylactic effect in the human or lower animal subject to whom the active is administered, without undue adverse side effects (such as toxicity, irritation, or allergic response), commensurate with a reasonable risk/benefit ratio when used in the manner of this invention. The specific safe and effective amount of the active will vary with such factors as the particular condition being treated, the physical condition of the subject, the nature of concurrent therapy (if any), the specific active used, the specific dosage form, the carrier employed, and the desired dosage regimen.

In some embodiments, the oral care compositions may include one or more abrasives or an abrasive system including one or more abrasives. As used herein, the term “abrasive” may also refer to materials commonly referred to as “polishing agents”. Any orally acceptable abrasive may be used, but preferably, type, fineness (particle size), and amount of the abrasive may be selected such that the tooth enamel is not excessively abraded in normal use of the oral care composition. The one or more abrasives may have a particle size or D50 of less than or equal to about 10 μm, less than or equal to about 8 μm, less than or equal to about 5 μm, or less than or equal to about 3 μm. The one or more abrasives may have a particle size or D50 of greater than or equal to about 0.01 μm, greater than or equal to about 0.05 μm, greater than or equal to about 0.1 μm, greater than or equal to about 0.5 μm, or greater than or equal to about 1 μm. Illustrative abrasives may include, but are not limited to, metaphosphate compounds, phosphate salts (e.g., insoluble phosphate salts), such as sodium metaphosphate, potassium metaphosphate, calcium pyrophosphate, magnesium orthophosphate, trimagnesium orthophosphate, tricalcium phosphate, dicalcium phosphate dihydrate, anhydrous dicalcium phosphate, calcium carbonate (e.g., precipitated calcium carbonate and/or natural calcium carbonate), magnesium carbonate, hydrated alumina, silica, zirconium silicate, aluminum silicate including calcined aluminum silicate, polymethyl methacrylate, or the like, or mixtures and combinations thereof. In some embodiments, the oral care composition comprises a silica abrasive. In some embodiments, the silica abrasive is present in an amount of from 10% to 30%, e.g., 10% to 20%, 15% to 25%, or about 15%, by weight, based on the total weight of the oral care composition. In some embodiments, the oral care composition comprises a calcium-free silica abrasive. In some embodiments, the composition is substantially free of calcium compounds. e.g., comprises less than 2%, less than 1%, less than 0.5%, or less than 0.1% of calcium compounds, by weight, based on the total weight of the oral care composition.

The oral care composition may include one or more agents to increase the amount of foam that is produced when the oral cavity is brushed. Such foaming agents are known to those of skill in the art. Illustrative examples of agents that increase the amount of foam include, but are not limited to polyoxyethylene and certain polymers including, but not limited to, alginate polymers. The polyoxyethylene may increase the amount of foam and the thickness of the foam. Polyoxyethylene is also commonly known as polyethylene glycol (“PEG”) or polyethylene oxide. The polyoxyethylenes suitable for this invention will have a molecular weight of 200,000 to 7,000,000, e.g., 600,000 to 2,000,000 or 800.000 to 1,000,000. The polyoxyethylene may be present in an amount of 1% to 90%, e.g., 5% to 50% or 10% to 20%, by weight, based on the total weight of the oral care composition. The dosage of foaming agent in the composition (i.e., a single dose) is 0.01 to 0.9%, e.g., 0.05 to 0.5% or 0.1 to 0.2%, by weight, based on the total weight of the oral care composition.

The oral care composition may include at least one surfactant or solubilizer. Suitable surfactants include neutral surfactants (such as polyoxyethylene hydrogenated castor oil or fatty acids of sugars), anionic surfactants (such as sodium lauryl sulfate), cationic surfactants (such as the ammonium cation surfactants) or zwitterionic surfactants. These surfactants or solubilizers may be present in amounts of typically 0.01% to 2%; or from 1% to 2%; or about 1.5%, by weight, based on the total weight of the oral care composition.

The oral care composition of the present invention may include a sweetener such as, for example, saccharin, for example sodium saccharin, acesulfame, neotame, cyclamate or sucralose; natural high-intensity sweeteners such as thaumatin, stevioside or glycyrrhizin; or such as sorbitol, xylitol, maltitol or mannitol. One or more of such sweeteners may be present in an amount of from 0.005% to 5% by weight, for example 0.01% to 1%, for example 0.01% to 0.5%, by weight, based on the total weight of the oral care composition.

The oral care composition may include one or more colorants. Colorants may include pigments, dyes, lakes and agents imparting a particular color or visual quality to the composition. Any orally acceptable colorant can be used. One or more colorants may optionally be present in the compositions in an amount of from 0.001% to 2%, for example from 0.001% to 0.01%, for example from 0.001% to 0.005%, by weight, based on the total weight of the oral care composition by weight.

The oral care composition may include one or more humectants. Humectants can reduce evaporation and also contribute towards preservation by lowering water activity, and can also impart desirable sweetness or flavor to compositions. Suitable humectants include edible polyhydric alcohols such as glycerin, sorbitol, xylitol, propylene glycol as well as other polyols and mixtures of these humectants. Other useful materials may also include orally acceptable alcohols, or polymers, e.g., such as polyvinylmethyl ether maleic acid copolymers, polysaccharides (e.g. cellulose derivatives, for example carboxymethyl cellulose, or polysaccharide gums, for example xanthan gum or carrageenan gum). In some embodiments, the humectant can be present in an amount of from 20% to 60%, for example from 30% to 50%, for example from 40% to 45%, by weight, based on the total weight of the oral care composition.

The composition of the present invention may include a preservative. Suitable preservatives include, for example, sodium benzoate, potassium sorbate, methylisothiazolinone, paraben preservatives, for example methyl p-hydroxybenzoate, propyl p-hydroxybenzoate, and mixtures thereof.

The composition of the present invention may include a flavoring agent. Suitable flavoring agents include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. The flavoring agent is typically incorporated in the oral composition at a concentration of 0.01 to 3% by weight based on the total weight of the oral care composition.

The oral care compositions can be manufactured following standard formulation procedure. For example, the toothpaste compositions can be manufactured as follows. Polymer gums are dispersed in glycerin with gentle stirring to make completely homogeneous gel phase. A premix is prepared by dissolving stannous fluoride and sodium saccharin in formula amounts of water. The premix solution is added to the gel phase and mixed for 12-15 minutes. Potassium nitrate, Chelator (citrate or EDTA), antioxidant (quercetin and catechol), tetrasodium pyrophosphate (TSPP), silica, zinc oxide and/or titanium dioxide are added to the mixture and mixed at low speed for 3-5 minutes for proper mixing. The mixture is then mixed at an increased speed under vacuum for 25-30 minutes to create a smooth dentifrice. Surfactants and flavoring agents are added to the composition and mixed at full speed under vacuum for 12-15 minutes until homogeneous.

In another aspect, the present invention provides a method to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity, comprising applying an effective amount of any of oral care composition (e.g., a dentifrice composition) as disclosed herein to the oral cavity of a subject in need thereof.

In another aspect, the present invention provides a method to improve oral health comprising applying an effective amount of any of oral care compositions as disclosed herein to the oral cavity of a subject in need thereof.

In another aspect, the present invention provides the use of any of oral care compositions as disclosed herein to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity, in a subject in need thereof.

In another aspect, the invention provides the use of a chelator or an antioxidant in an oral care composition comprising a stannous ion source for increasing the stability of stannous ion in the composition.

The following examples are further illustrative of the preferred embodiments, but it is understood that the invention is not limited thereto.

### EXAMPLES

### Example 1

Two samples of Sn(II) soluble complexes (hereinafter for simplicity referred to as Sn(II)-EDTA (1:1 molar ratio) and Sn(II)-citrate (1:2 molar ratio)) were prepared in this study. Table 1 lists the amounts of raw materials used in preparation of solutions.

TABLE 1
Raw materials and their quantities used in preparation of samples

| | Sn(II)- | Sn(II)- | Sn(II)- | Sn(II)- |
|---|---|---|---|---|
| | EDTA | EDTA w/ | citrate | citrate w/ |
| Reagent | (g) | KNO₃ (g) | (g) | KNO₃ (g) |
| SnF₂ | 0.504 | 0.503 | 0.397 | 0.404 |
| H₄EDTA | 0.948 | 0.938 | — | — |
| KNO₃ | — | 0.325 | — | 0.26 |
| Na₃Cit*2H₂O | — | — | 1.44 | 1.512 |
| H₂O | 23.54 | 23.23 | 18.63 | 18.61 |

Na₃Cit*2H₂O = HOC(COONa)(CH2COONa)₂•2H₂O

The pH of all the solutions was adjusted to pH=7 using HCl or NaOH. The solutions were not fully clear. Small amount of the precipitate/powder was evident at the bottom of the vials. All the measurements were performed on the supernatant part.

Fourier transform infrared spectroscopy (FTIR): Infrared spectra were collected using a Bruker Vertex 70 FTIR spectrometer (Bruker Optics, Billerica, MA) equipped with a GladiATR diamond ATR accessory (Pike technologies, Madison, WI). The spectral range was 80-4000 cm-1 and a resolution of 4 cm⁻¹ was used. All measurements were carried out at room temperature on as prepared samples.

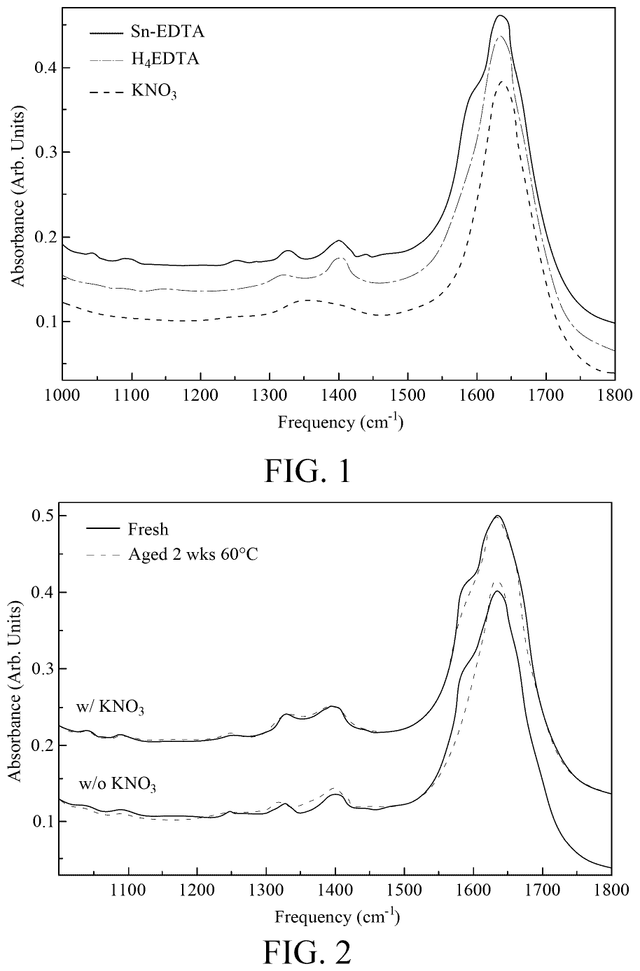

FIG. 1 shows a close-up view of the infrared absorption spectrum of Sn(II)-EDTA complex (Table 1) along with the spectra of EDTA and KNO₃ solutions. In the presence of tin, several EDTA bands exhibited frequency shifts and intensity changes, indicating the formation of the Sn(II)-EDTA complex. The most pronounced change was observed in the 1570 cm⁻¹ peak attributed to the asymmetric stretching vibration of the carboxylate groups (νₐₛ(COO⁻)). The peak can thus be used as one of the indicators of complex stability with aging. The vibrational spectra of Sn(II)-EDTA complex in the absence or presence of KNO₃ at different time points, fresh and 2 weeks aged at a temperature of 60° C. are presented in FIG. 2. EDTA bands of Sn(II)-EDTA complex without KNO₃ exhibited pronounced changes with aging. After 2 weeks at a temperature of 60° C. the v.(COO⁻) band showed a pronounced drop in intensity and/or blueshift. In contrast, Sn(II)-EDTA complex in the presence of KNO₃ showed more modest changes in the EDTA peaks with aging. This indicates that Sn(II)-EDTA complex is more stable in presence of KNO₃, thus suggesting that KNO₃ slows down the oxidation of Sn(II) to Sn(IV) species.

The vibrational spectra of Sn(II)-citrate complex in the absence or presence of KNO₃ at different aging times: fresh, 1 week at a temperature of 60° C. and 2 weeks at a temperature of 60° C. were also examined. The citrate vibrational bands did not exhibit any substantial changes with aging in the absence or presence of KNO₃. This suggests that citrate environment does not change significantly upon Sn(II) oxidation or that Sn(II)-Citrate complex is stable with aging. In order to address this question, Nuclear Magnetic Resonance (NMR) measurements were performed to look directly at stannous behavior.

Nuclear Magnetic Resonance (NMR): NMR measurements were performed on samples in the presence of 10% deuterium oxide (D₂O). All NMR spectra were acquired on a Bruker Avance spectrometer (Bruker-Biospin, Billerica, MA, USA) with a 5 mm liquid nitrogen cryogenic probe operating at 500.1 MHz for ¹H, and 186.5 MHz 129Sn at a temperature of 25° C. For ¹H NMR, a solvent saturation pulse sequence was used to suppress the water peak.

¹¹⁹Sn NMR spectra of Sn(II)-citrate complex in the absence or presence of KNO₃ at different aging times: fresh, 1 week at a temperature of 60° C. and 2 weeks at a temperature of 60° C. were examined. For the SnF₂+citrate sample (i.e., Sn(II)-citrate complex), only a peak near 580 ppm corresponding to Sn(II) was present in the fresh sample. After the sample without KNO₃ was aged for 1 week and 2 weeks at a temperature of 60° C., two small peaks between 600 and 630 ppm corresponding to Sn(IV) started to show up while Sn(II) peak intensity slightly decreased. This indicates that Sn(II) was partially oxidized to Sn(IV). However, when KNO₃ was present in SnF₂+citrate solution, Sn(II) signal exhibited a more stable behavior and did not have significant decrease in intensity upon aging.

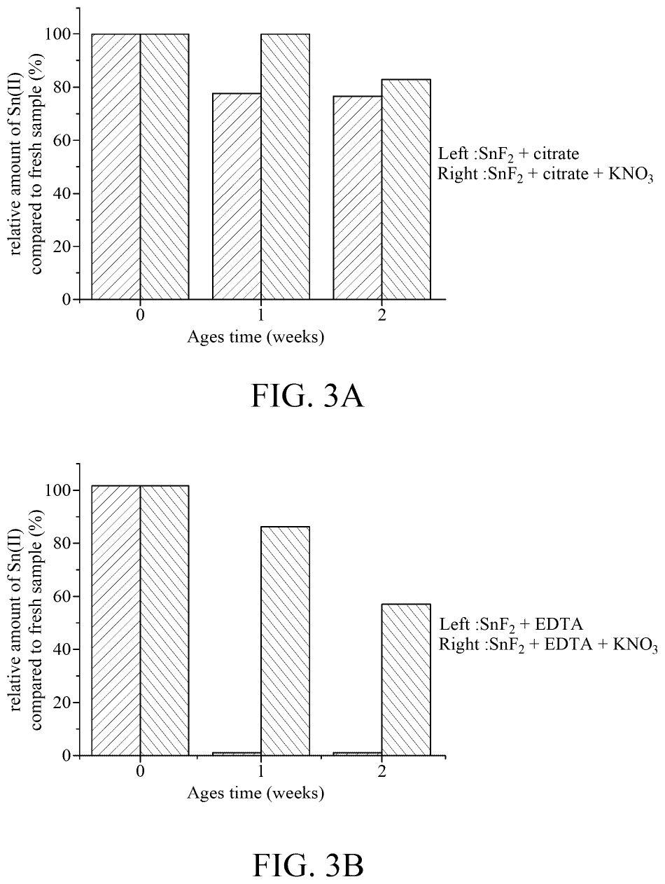

¹¹⁹Sn NMR spectra of Sn(II)-EDTA complex in the absence or presence of KNO₃ at different aging times: fresh, 1 week at a temperature of 60° C. and 2 weeks at a temperature of 60° C. were also examined. For the SnF₂+EDTA sample in the absence of KNO₃, Sn(II) completely oxidized after 1 week of aging, while Sn(II) in SnF₂+EDTA sample in the presence of KNO₃ still had 80% Sn(II). The relative amounts of Sn(II) compared to fresh sample were calculated from the integrated area of the peak corresponding to Sn(II) in ¹¹⁹Sn NMR spectra. The results are shown in FIGS. 3A and 3B. These results show that the presence of KNO₃ can stabilize Sn(II) in the Sn(II)-citrate or Sn-EDTA complex at neutral pH.

Head-space O₂ consumption: Stannous oxidation reactions were carried out in a closed 250 mL round-bottom flask and were monitored by pressure changes in the gaseous (air) headspace above the solution at a constant temperature of 25 t 0.5° C. In a typical experiment the vessel was filled with 100 mL of solution containing 29 mM SnF₂ and 58 mM HOC(COONa)(CH₂COONa)₂·2H₂O, with or without 29 mM KNO₃. SnF₂ was added last as a powder, and the solution was immediately sealed with a glass flask stopper. The solution was stirred magnetically at 750 rpm and the differential pressure was recorded over about 6 hour period. Digital manometer (APT Instruments, MP2000) connected to the flask was used to record the differential pressure. The pH of the solution was adjusted to 6.5 and did not exhibit significant changes during the reaction.

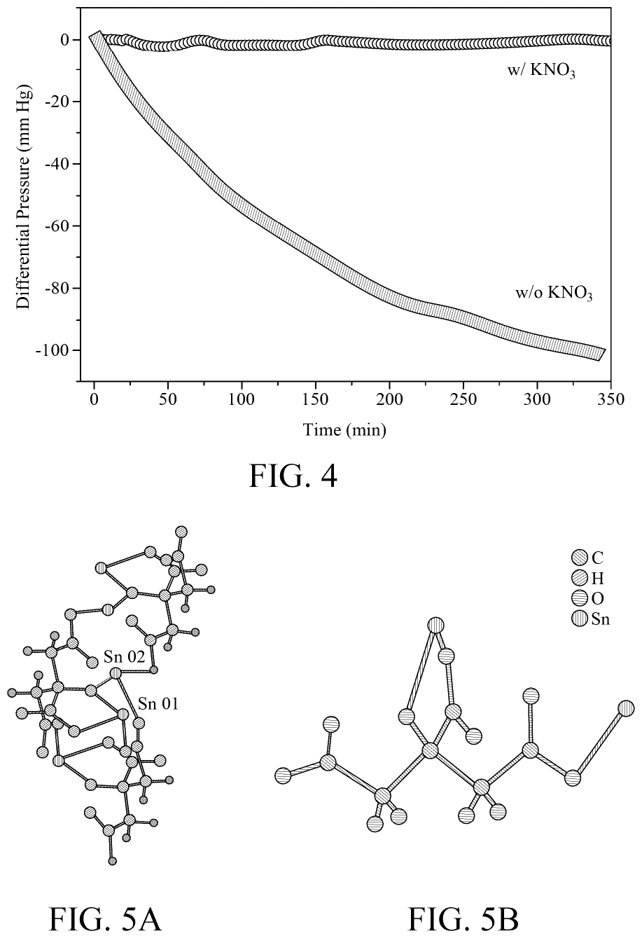

The progress of the stannous oxidation reaction was monitored through gas-phase pressure changes above the solution of interest reflecting the consumption of oxygen during the reaction. FIG. 4 shows the differential pressure readings as a function of time for solutions containing stannous fluoride and sodium citrate with or without KNO₃. In the absence of KNO₃, a continuous drop in pressure was observed over the 6 hours of data collection, indicating a rapid consumption of O₂ due to stannous oxidation process. In contrast, when KNO₃ is present in the same solution, no significant pressure drop was observed over the same period of time, confirming the suppressing effect of KNO₃ on the oxidation kinetics of stannous.

### Example 2: Stannous Citrate Crystal

Citric acid (0.192 g, 1 mmol) was dissolved in water (10 mL) and the solution was frozen and degassed three times to create an inert environment. After freezing the solution again, SnCl₂·2H₂O (0.450 g, 2 mmol) was added into the flask, and then degassed and filled with N₂. Upon reaching room temperature, the solution was then heated to a temperature of 80° C. for 2 h. After cooling down, the solution was placed into a refrigerator overnight and colorless tiny sheet-like crystals were observed. Single crystal X-ray diffraction (SCXRD) data for the Sn-Citrate crystal were measured on Bruker D8 Venture PHOTON II CPAD diffractometer equipped with a Cu Kα INCOATEC ImuS micro-focus source (λ=1.54178 Å). Indexing was performed using APEX3. Data integration and reduction was performed using SaintPlus. Absorption correction was performed by multi-scan method implemented in SADABS. Space group was determined using XPREP implemented in APEX3. Structure was solved using SHELXT and refined using SHELXL-2019 (full-matrix least-squares on F2) through Olex2.

Single crystal X-ray diffraction (SCXRD) data show that Sn-citrate crystallizes in monoclinic C2/c space group with the unit cell parameters of a=17.23 Å, b=7.57 Å and c=18.71 Å with a unit cell volume of 2278.29 Å³. The structural formula of the asymmetric unit can be established as [Sn₂(C₆H₄O₇)(H₂O)₃]ₐ (hereinafter referred to as tin-citrate) which consists of two Sn(II) metal centers bridged by the carboxylate oxygen atoms from one citrate and the hydroxyl oxygen atom from another citrate forming a m₂-oxo cluster. The hydroxyl oxygen and the carboxylate oxygen of the second citrate chelate to one of the Sn(II) centers while the other Sn(II) center coordinates to the carboxylate oxygen of a third citrate molecule (FIG. 5A). The uncoordinated solvent oxygen molecules were modeled as H₂O. Each citrate on the other hand coordinates to four different Sn(II) centers (FIG. 5B); one of the carboxylate bridges between two Sn(II) centers and the second carboxylate on the other end coordinates to one Sn(II). The third carboxylate along with the hydroxyl group from the same carbon center chelates one Sn(II) and finally the hydroxyl oxygen coordinates to another Sn(II) forming a one-dimensional chain. The presence of hydrogen bonding interactions between the Sn(II) center of one chain and oxygen atom of the carboxylate atom from the second chain (2.670 Å) lead to the formation of a supramolecular 2-D coordination network. The presence of uncoordinated solvent water molecules further strengthens the hydrogen bonding interactions between the chains. This type of coordination networks based on Sn(II) are very rare in the literature and since Sn(II) is not fully coordinated, these can be further exploited to form extended networks.

After synthesis of crystals, the suspension was allowed to settle down under the N₂ atmosphere. The upper layer was then taken out by syringe and new DI water was injected. This procedure was repeated 3 times to wash off most of free ions in the mixture. The deposit was divided into equal portions and water or KNO₃ solution was injected (mole ratio KNO₃: tin-citrate=1.2:1). The weight of tin-citrate was obtained by drying the sample in an inert atmosphere. The suspensions were then exposed to air for testing the stability. The samples for stability tests were obtained by vacuum filtration of the suspensions at certain time points. The stability of the tin-citrate was characterized by evaluation of the PXRD (Powder X-ray Diffraction) patterns over a period of time. The stability test data were generated by comparing PXRD patterns of the treated sample with the pristine tin-citrate material. All testing was done at room temperature.

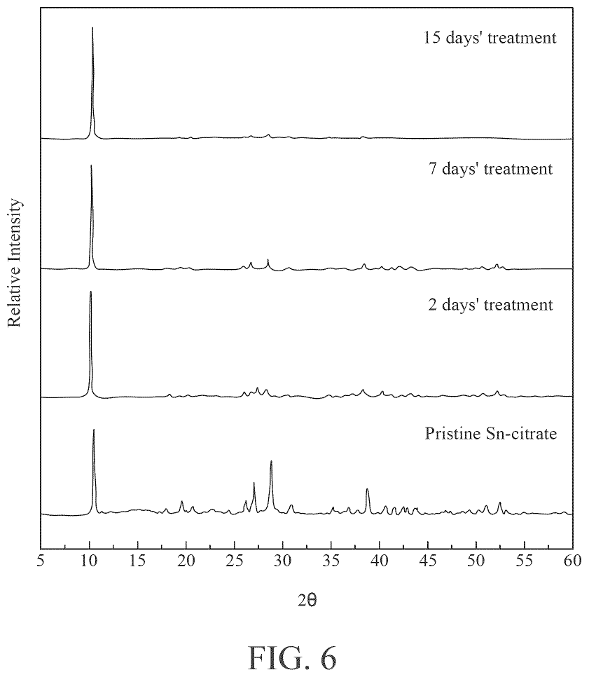

Without KNO₃ treatment: After 2 days, one portion of sample was taken out and separated by vacuum filtration. The sample was then immediately analyzed using a quick scan of the patterns. Same procedures were also applied to the sample after 7 and 15 days. FIG. 6 shows PXRD of tin-citrate samples soaked in deionized water. As evident from FIG. 6, the strength of peaks within 20-55° 2theta decreased continuously and finally most peaks disappear, implying that the tin-citrate decomposes over a period of two weeks.

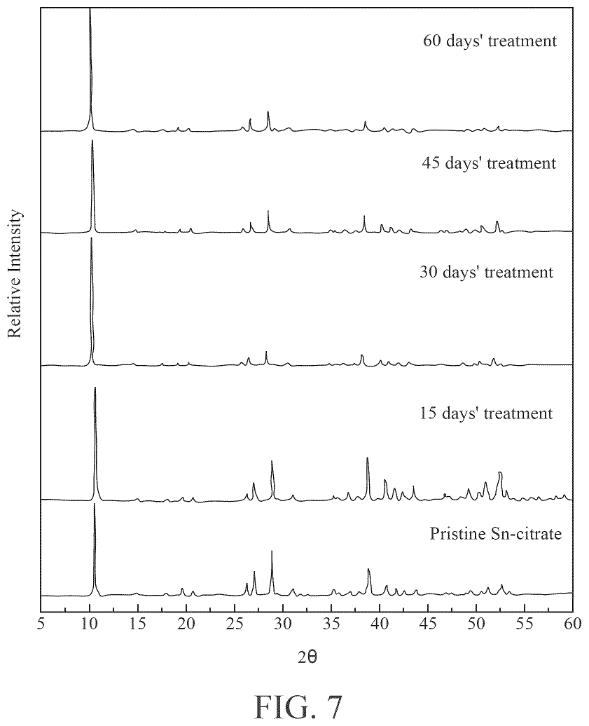

With KNO₃ treatment: The same procedures were applied for the stability evaluation of the samples that were treated with KNO₃ solution. FIG. 7 shows PXRD of tin-citrate samples soaked in KNO₃ solution. In this case the strength of peaks in the 20-55° 2theta range was retained perfectly after 15 days of treatment. The stability tests were then extended to 30, 45 and 60 days and the peaks are still retained even after 60 days with a slight decrease in the intensity, indicating that the tin-citrate is stabilized in the presence of KNO₃. These data demonstrate that the addition of KNO₃ to aqueous solution results in improved stability of tin-citrate crystal and suggest reduced oxidation of Sn(II), the most common reason for the degradation of stannous complexes. This work indicates that tin(II)-chelator stabilization by KNO₃ is achievable in both soluble and solid state.

### Example 3

The effect of antioxidants on the stannous stability in the presence of tetrasodium pyrophosphate (TSPP) was examined. Samples of Sn(II) were prepared by dissolving TSPP and SnF₂ in water with the final concentration of SnF₂ equal to 2 wt. %. Antioxidant (quercetin or catechol) was added to the samples with the molar ratio of SnF₂:TSPP:antioxidant equal to 1:1:0.3. The pH of the solutions was adjusted with NaOH to pH=7. The solutions were not fully clear due to the high concentration of SnF₂ and limited solubility of quercetin. Some amount of the precipitate/undissolved material was evident in the vials. Table 2 lists the amounts of raw materials used in preparation of solutions.

TABLE 2
Raw materials and their quantities used in preparation of samples

| | SnF₂ | TSPP | Antioxidant | H₂O | |
|---|---|---|---|---|---|
| Sample | (g) | (g) | (g) | (g) | pH |
| SnF₂-TSPP-Quercetin | 2.0030 | 3.3950 | 1.1170 | 93.4025 | 7.02 |
| SnF₂-TSPP-Catechol | 2.0018 | 3.3930 | 0.4220 | 94.1857 | 7.01 |

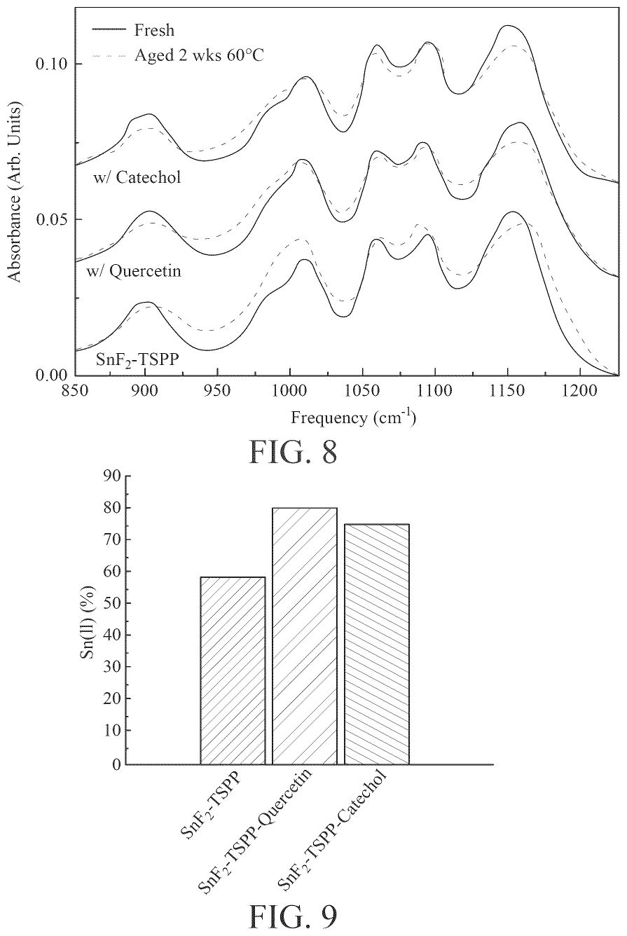

Fourier transform infrared spectroscopy (FTIR): Infrared spectra were collected using a Bruker Vertex 70 FTIR spectrometer (Bruker Optics, Billerica, MA) equipped with a GladiATR diamond ATR accessory (Pike technologies, Madison, WI). The spectral range was 80-4000 cm⁻¹ and a resolution of 4 cm⁻¹ was used. All measurements were carried out at room temperature. The absorption spectrum of solution containing SnF₂ and TSPP (i.e., SnF₂-TSPP) solution in the 850-1225 cm⁻¹ region at different time points: fresh and 2 weeks aged at a temperature of 60° C. was examined. The FTIR spectra of solutions containing SnF₂ and TSPP with and without antioxidant are shown in FIG. 8. Clear changes in the pyrophosphate bands of the stannous-pyrophosphate complex were observed with aging. The transformation of the complex was manifested in the peak shifts and intensity changes of phosphate bands. As an example, a doublet with peaks near 1058 and 1094 cm⁻¹ exhibited an inward band shifting towards a greater overlap of the two features after 2 weeks at a temperature of 60° C. When quercetin or catechol was added to stannous solution, the doublet as well as other phosphate absorption bands displayed more modest changes with aging, indicating better stability of stannous-pyrophosphate complex and slower Sn(II) oxidation in the presence of quercetin or catechol.

Iodine titration: The stability of Sn (II) was further examined by iodine titration assay. Titration measurements were performed through indirect titration. 2 M citric acid solution was added to the samples in excess and 0.1 N Iodine was added until the solution turned light brown. The samples were covered with parafilm and aluminum foil and left mixing in the dark for 2 hours. Then, titration with 0.1 N sodium thiosulfate standard was performed using a 25 mL Titrette Bottletop Burette (BrandTech Scientific, Inc., Essex, CT, USA) until the solution turned clear. Sn(II) concentration was calculated based on the amount of sodium thiosulfate and iodine used in the reaction. The amount of Sn(II) present in the SnF₂-TSPP solution after 2 week of a temperature of 60° C. aging with and without the antioxidant (quercetin or catechol) was measured. The result is shown in FIG. 9. The result indicates that quercetin and catechol improve stannous stability in aqueous solution in the presence of TSPP.

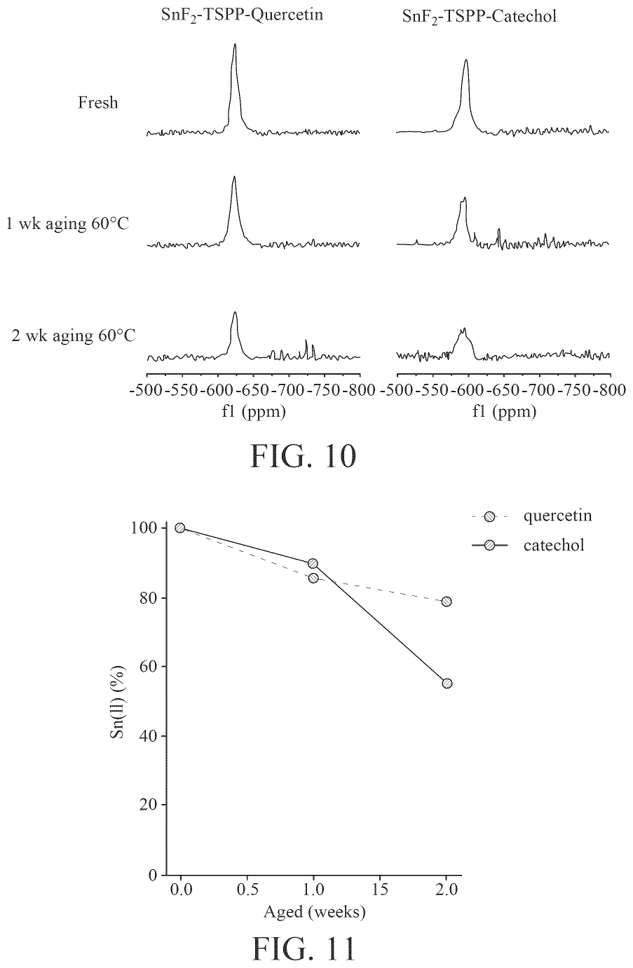

Nuclear Magnetic Resonance (NMR): The stability of Sn (II) was further examined by Nuclear Magnetic Resonance (NMR). NMR measurements were performed on samples in the presence of 10% deuterium oxide (D₂O). All NMR spectra were acquired on a Bruker Avance spectrometer (Bruker-Biospin, Billerica, MA, USA) with a 5 mm liquid nitrogen cryogenic probe operating at 202 MHz for ³¹P, and 186.5 MHz 129Sn at a temperature of 25° C. ¹¹⁹Sn spectra were used to quantify the Sn(II) content in the samples containing SnF₂, TSPP and quercetin (i.e., SnF₂-TSPP-Quercetin), and SnF₂. TSPP and catechol (i.e., SnF₂-TSPP-Catechol) after aging. FIG. 10 shows ¹¹⁹Sn NMR spectra of SnF₂-TSPP-Quercetin and SnF₂-TSPP-Catechol upon 2 weeks aging at a temperature of 60° C. The peak position at around −630 ppm corresponds to Sn(II) in solution at neutral pH. The Sn(II) peak intensity decreased on both samples upon aging at a temperature of 60° C., but SnF₂-TSPP-Quercetin solution had higher Sn(II) content than that of SnF₂-TSPP-Catechol sample. The relative amounts of Sn(II) compared to fresh sample were calculated from the integrated area of the peak corresponding to Sn(II) in ¹¹⁹Sn NMR spectra. The result is shown in FIG. 11. These data show that compared to SnF₂-TSPP solution that showed practically no Sn(II) signal in NMR after 2 weeks of a temperature 60° C. aging, both quercetin and catechol can stabilize Sn(II), consistent with the FTIR and titration result.

### Example 4

A non-limiting example oral care composition (Example Composition A) was prepared in in the form of a dentifrice in accordance with aspects of the invention. A comparative Composition (Comparative Composition 1) was also prepared in the form of a dentifrice. The formulations for Example Composition A and Comparative Composition 1 is shown in Table 3.

TABLE 3

| | Ex. A | Comp. 1 |
|---|---|---|
| Stannous Fluoride (wt. %) | 0.454 | 0.454 |
| Trisodium Citrate Dihydrate | 2.1 | 2.1 |
| (wt. %) | | |
| Potassium Nitrate (wt. %) | 0.5 | 0 |
| Molar Ratio of Stannous ion: | 1:2.5:1.7 | |
| Citrate:Nitrate ion | | |
| Sorbitol (wt. %) | 45 | 45 |
| Glycerin (wt. %) | 7.286 | 7.786 |
| Abrasive Silica (wt. %) | 10 | 10 |
| High Cleaning Silica (wt. %) | 10 | 10 |
| Thickening Silica (wt. %) | 4 | 4 |
| Xanthan Gum (wt. %) | 0.4 | 0.4 |
| Flavor, Colors, and other | 2.18 | 2.18 |
| Minors (wt. %) | | |
| Anionic surfactant (e.g. Sodium | 1.5 | 1.5 |
| Lauryl Sulfate) (wt. %) | | |
| Zwitterionic Surfactant (e.g. | 1.25 | 1.25 |
| Cocamidopropyl Betaine) | | |
| (wt. %) | | |
| Carboxymethyl Cellulose | 0.6 | 0.6 |
| (wt. %) | | |
| NaOH (wt. %) | 0.1 | 0.1 |
| Water (wt. %) | Q.S. (~15) | Q.S. (~15) |

Samples of Example Composition A and Comparative Composition 1 were evaluated to assess the stability of the stannous ion source. Specifically, stannous oxidation reactions were carried out in a closed 250 mL round-bottom flask and were monitored by pressure changes in the gaseous headspace (air) above the solution at a constant temperature of 25±0.5° C. In a typical experiment toothpaste was diluted with water (33.3 g toothpaste: 66.7 g water), stirred gently with a plastic spatula to achieve a homogeneous slurry and transferred to a flask. The flask was sealed with a rubber septum. The solution was constantly stirred magnetically and the differential pressure was recorded over about a 24 hour period. Digital manometer (APT Instruments, MP2000) connected to the flask was used to record the differential pressure.

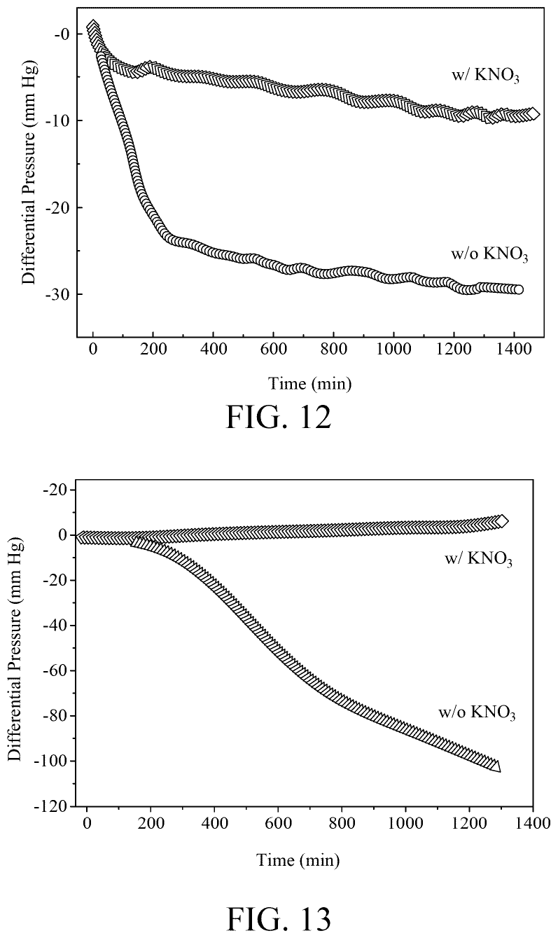

The progress of the stannous oxidation reaction was monitored through gas-phase pressure changes above the toothpaste solution of interest reflecting the consumption of oxygen from the air during the reaction. FIG. 12 is a graph showing the differential pressure readings as a function of time for solutions of Example Composition A and Comparative Composition 1. Initially, both samples display a similar rate of oxygen consumption; however, after about 15 mins pressure drop in the toothpaste sample containing nitrate (1) starts to slow down, while the differential pressure in the sample with no nitrate (2) ion continues to drop indicating a rapid consumption of 0₂ due to stannous oxidation process.

### Example 5

A non-limiting example solution having stannous fluoride, EDTA, and potassium nitrate and a comparative solution having stannous fluoride and EDTA, but no potassium nitrate were prepared. The example solution and the comparative solution were evaluated to assess the stability of the stannous ion source. Specifically, stannous oxidation reactions were carried out in a closed 250 mL round-bottom flask and were monitored by pressure changes in the gaseous headspace (air) above the solution at a constant temperature of 25 t 0.5° C. In a typical experiment the vessel was filled with 100 mL of solution containing 29 mM SnF₂, 29 mM EDTA and 29 mM KNO₃. SnF₂ was added last as a powder, and the flask was immediately sealed with a rubber septum. The solution was stirred magnetically at 750 rpm and the differential pressure was recorded over about a 22 hour period. Digital manometer (APT Instruments, MP2000) connected to the flask was used to record the differential pressure. The pH of the solution was adjusted to 7 and did not exhibit significant changes during the reaction.

The progress of the stannous oxidation reaction was monitored through gas-phase pressure changes above the solution of interest reflecting the consumption of oxygen from the air during the reaction. FIG. 13 is a graph showing the differential pressure readings as a function of time for solutions containing stannous fluoride and EDTA with and without nitrate. In the absence of KNO₃ a significant drop in pressure was observed over the 22 hours of data collection, indicating a rapid consumption of O₂ due to stannous oxidation process. In contrast, when nitrate is present in the same solution, no significant pressure drop was observed over the same period of time, emphasizing the suppressing effect of nitrate ion on the oxidation kinetics of stannous.

1. An oral care composition comprising: (i) a stannous ion source and (ii) a chelator or an antioxidant.

2. The oral care composition of claim 1, wherein the chelator is selected from citrate and EDTA and the composition comprises potassium nitrate.

3. The oral care composition of claim 2, wherein the chelator is citrate.

4. The oral care composition of claim 3, wherein the molar ratio of stannous ion source to citrate to potassium nitrate is 1:1.5-2.5:0.5-1.5.

5. The oral care composition of claim 2, wherein the chelator is EDTA.

6. The oral care composition of claim 5, wherein the molar ratio of stannous:EDTA:potassium nitrate is 1:0.5-1.5:0.5-1.5.

7. The oral care composition of claim 1, wherein the oral care composition does not contain polyphosphate or pyrophosphate.

8. The oral care composition of claim 1, wherein the oral care composition comprises an antioxidant selected from quercetin and catechol.

9. The oral care composition of claim 8, wherein the oral care composition comprises tetrasodium pyrophosphate (TSPP).

10. The oral care composition of claim 9, wherein the oral care composition has a molar ratio of stannous ion source to TSPP to antioxidant of 1:0.5-1.5:0.2-0.4.

11. The oral care composition of claim 1, wherein the stannous ion source is selected from the group consisting of stannous fluoride, stannous gluconate, stannous phosphate, stannous pyrophosphate, stannous acetate, stannous sulfate, stannous chloride and a combination thereof.

12. The oral care composition of claim 1, wherein the stannous ion source is present in an amount of from 0.1% to 5%, by weight, based on the total weight of the oral care composition.

13. The oral care composition of claim 1, wherein the oral care composition comprises a fluoride ion source.

14. The oral care composition of claim 1, wherein the oral care composition comprises a zinc ion source.

15. The oral care composition of claim 1, wherein the oral care composition comprises a basic amino acid.

16. The oral care composition of claim 1, wherein pH of the oral care composition is from 6.5 to 7.5.

17. The oral care composition of claim 1, wherein the oral care composition is a dentifrice, a toothpaste, a gel, or a mouthwash.

18. (canceled)

19. A method of (i) reducing or inhibiting formation of dental caries, (ii) reducing, repairing or inhibiting pre-carious lesions of the enamel, (iii) reducing or inhibiting demineralization and promoting remineralization of the teeth, (iv) reducing hypersensitivity of the teeth, (v) reducing or inhibiting gingivitis, (vi) promoting healing of sores or cuts in the oral cavity, (vii) reducing levels of acid producing bacteria, (viii) reducing or inhibiting microbial biofilm formation in the oral cavity, (ix) reducing or inhibiting plaque formation in the oral cavity, (x) promoting systemic health, or (xi) cleaning teeth and oral cavity, comprising applying an oral care composition according claim 1 to the oral cavity.

20. (canceled)
